(19) United States
(12) Patent Application Publication
(10) Pub. No.: US 2023/0063218 A1
(43) Pub. Date: Mar. 2, 2023

(54) MEDICAL INFORMATION PROCESSING APPARATUS

(71) Applicant: Canon Medical Systems Corporation, Otawara-shi (JP)

(72) Inventors: Yudai YAMAZAKI, Nasushiobara (JP); Kazumasa NORO, Shioya (JP); Sho SASAKI, Utsunomiya (JP); Kosuke ARITA, Otawara (JP)

(73) Assignee: Canon Medical Systems Corporation, Otawara-shi (JP)

(21) Appl. No.: 17/821,208

(22) Filed: Aug. 22, 2022

(30) Foreign Application Priority Data
Aug. 27, 2021 (JP) 2021-138704

(51) Int. Cl. G16H 15/00; G16H 70/60

(52) U.S. Cl. CPC G16H 15/00; G16H 70/60

(57) ABSTRACT

According to one embodiment, a medical information processing apparatus includes a processing circuitry. The processing circuitry determines respective medical information pieces as display candidates based on a plurality of support information pieces, and respective weights of a plurality of medical information pieces. The processing circuitry decides whether or not to integrate the respective medical information pieces based on the respective medical information pieces as the display candidates, and the plurality of support information pieces. The processing circuitry determines an arrangement of medical information pieces based on the plurality of support information pieces as a result of the decision.

### CROSS-REFERENCE TO RELATED APPLICATION

This application is based upon and claims the benefit of priority from the prior Japanese Patent Application No. 2021-138704, filed Aug. 27, 2021, the entire contents of which are incorporated herein by reference.

### FIELD

Embodiments described herein relate generally to a medical information processing apparatus.

### BACKGROUND

In comprehensive medical care, a doctor determines a disease of a target patient and decides a treatment policy therefor based on patient's symptoms and past examination results. Here, a doctor is required to consider the possibility of various diseases at all times in a scene of identifying the disease of a patient and a scene of grasping the condition of a patient who has been hospitalized due to an identified disease such as cancer. For example, in the field of cancer, an initial stage is a state in which a cancer stays in a specific organ, but metastasis may spread the cancer to different organs. Further, it is said that treatments (chemotherapy and radiation therapy) for cancer affects the heart, which increases a risk of contracting heart disease.

In recent years, a CDS (clinical decision support) system for presenting support information such as a disease risk and medical information forming a basis for the support information to users such as doctors has been researched and developed. For a plurality of support information pieces, the CDS makes it possible to present respective related medical information pieces to a user on the time axis of the same period to promote the user to judge the validity of each support information piece, thereby supporting identifying a disease and deciding a treatment policy. Usually, such CDS is not particularly problematic, but according to the studies of the inventor of the present application, medical information off the time axis of the same timing period may not be displayed in a limited display range. In other words, there is room for improvement in the CDS in that related medical information may not be presented to the user depending on the timing period of the medical information.

### BRIEF DESCRIPTION OF THE DRAWINGS

FIG. 1 is a block diagram showing a medical information processing apparatus according to a first embodiment and a peripheral configuration thereof.

FIG. 2 is a block diagram showing a configuration of the medical information processing apparatus according to the first embodiment.

FIG. 3 is a schematic diagram describing a support information table in the first embodiment.

FIG. 4 is a schematic diagram describing a weight table in the first embodiment.

FIG. 5 is a schematic diagram describing a display candidate table in the first embodiment.

FIG. 6 is a schematic diagram describing an integration target table in the first embodiment.

FIG. 7 is a flowchart describing an operation in the first embodiment.

FIG. 8 is a flowchart describing an operation shown in step S30 of FIG. 7.

FIG. 9 is a flowchart describing an operation shown in step S40 of FIG. 7.

FIG. 10 is a schematic diagram describing an operation shown in step S45 of FIG. 9.

FIG. 11 is a flowchart describing an operation shown in step S50 of FIG. 7.

FIG. 12 is a schematic diagram showing an example of a display screen to describe an operation shown in step S60 of FIG. 7.

FIG. 13 is a schematic diagram showing another example of the display screen to describe the operation shown in step S60 of FIG. 7.

FIG. 14 is a schematic diagram showing still another example of the display screen to describe the operation shown in step S60 of FIG. 7.

FIG. 15 is a schematic diagram describing an optimization target table used in a medical information processing apparatus according to a second embodiment.

FIG. 16 is a flowchart describing an operation of step S40 in the second embodiment.

FIG. 17 is a schematic diagram describing an operation shown in step S45a of FIG. 16.

FIG. 18 is a flowchart describing an operation of step S50 in the second embodiment.

FIG. 19 is a schematic diagram describing an operation shown in step S52a of FIG. 18.

FIG. 20 is a schematic diagram showing an example of a display screen to describe an operation shown in step S60 in the second embodiment.

### DETAILED DESCRIPTION

In general, according to one embodiment, a medical information processing apparatus includes a processing circuitry. The processing circuitry is configured to determine respective medical information pieces as display candidates based on a plurality of support information pieces which individually include index values related to a plurality of diseases, and respective weights of a plurality of medical information pieces related to the respective index values related to the plurality of diseases. The processing circuitry is configured to decide whether or not to integrate the respective medical information pieces based on the respective medical information pieces as the display candidates, and the plurality of support information pieces. The processing circuitry is configured to determine an arrangement of medical information pieces to be integrated and an arrangement of medical information pieces to be non-integrated based on the plurality of support information pieces as a result of the decision.

Respective embodiments will be described hereunder with reference to the drawings.

### First Embodiment

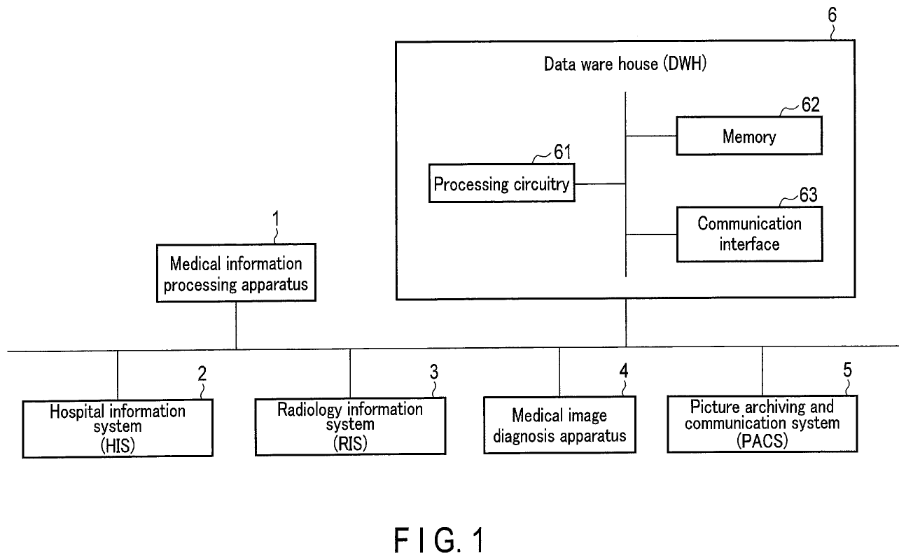

FIG. 1 is a block diagram showing a medical information processing apparatus according to a first embodiment and a peripheral configuration thereof. The medical information processing apparatus 1 shown in FIG. 1 is, for example, an apparatus that enables integrated observation of medical information. For example, an integrated viewer is installed in the medical information processing apparatus 1. The integrated viewer is an application for integrally presenting medical information to users. The integrated viewer may adopt any implementation form such as a Web application, a fat client application, or a thin client application. The medical information processing apparatus 1 is communicably connected to an HIS (Hospital Information System) 2, an RIS (Radiology Information System) 3, a medical image diagnosis apparatus 4, a PACS (Picture Archiving and Communication System) 5, and a DWH (Data Ware House) 6 via an intra-hospital network such as a LAN (Local Area Network).

In FIG. 1, HIS 2 comprises, for example, an electronic health record system for managing information regarding electronic health records. Information about an electronic health record includes, for example, patient information and a plurality of medical information pieces. The patient information is information unique to a patient, and includes, for example, a patient ID, patient name, sex, age, and the like.

The plurality of medical information pieces are information pieces which are associated with the patient ID, and can be known by a medical worker about a physical status of a patient, a disease, a medical treatment, and the like in the process of medical treatment. The plurality of medical information pieces may include the following information explained below as clinical data or medical data. Each of the plurality of medical information pieces individually includes, for example, various types of information such as image information, examination history information, electrocardiogram information, vital sign information, medication history information, report information, medical chart description information, and nursing record information. The various types of information in the medical information can be distinguished from one another according to the data type. Similarly, various types of information contained in each of image information, examination history information, electrocardiogram information, vital sign information, medication history information, report information, medical chart description information, nursing record information, and the like can also be distinguished according to the data type. The image information is, for example, information indicating the location of a medical image acquired by imaging a patient or the like. The image information includes, for example, information indicating the location of a medical image file described later, which is generated by the medical image diagnosis apparatus 4 as a result of performing an examination. The examination history information is, for example, information representing the history of examination results obtained by performing specimen examination, bacteria examination, and the like with respect to a patient. The electrocardiogram information is, for example, information concerning the electrocardiographic waveform measured from a patient. The vital sign information is, for example, basic information concerning the life of a patient. The vital sign information includes, for example, a pulse rate, respiration rate, oxygen concentration, body temperature, blood pressure, and level of consciousness. The medication history information is, for example, information representing the history of the amounts of medicines administered to a patient. The report information is, for example, information obtained by a reading doctor in a radiology department by diagnostically reading medical images such as X-ray images, CT images, MRI images, and ultrasonic images in response to an examination request from a medical treatment doctor in a diagnosis and treatment department and summarizing information about the condition and disease of the patient. The report information includes, for example, reading report information representing the reading report generated by a reading doctor by referring to a medical image file stored in the PACS 5. Note that report information is generally stored in the PACS 5, and hence the electronic health record system can display the report information by reading out the report information from the PACS 5.

The medical record description information is, for example, information input to an electronic health record by a medical treatment doctor or the like. The medical record description information includes, for example, a medical treatment record at hospital admission, the medical history of a patient, and a medication prescription history.

The nursing record information is, for example, information input to an electronic health record by a nurse or the like. The nursing record information includes a nursing record at hospital admission, and the like.

The information concerning an electronic health record includes, for example, examination execution information. The examination execution information is generated by the medical image diagnosis apparatus 4, which has executed an examination according to examination order information. The examination execution information is information representing the examination executed by the medical image diagnosis apparatus 4. The examination execution information includes an order number, examination UID (Unique ID), patient ID, modality type, imaging region, and imaging conditions. The examination UID is an identifier that can uniquely specify an examination. The modality type represents the modality used for imaging. The modality type includes, for example, “X-ray computed tomography apparatus”, “X-ray diagnosis apparatus”, “magnetic resonance imaging apparatus”, and “ultrasonic diagnosis apparatus”. The imaging region corresponds to an examination region included in examination order information. The imaging region includes, for example, abdomen, brain, and breast. The imaging condition includes a body posture, imaging direction, and use or non-use of a contrast medium.

The HIS 2 comprises, for example, an order system that manages reservation information, and order information. Note that the HIS 2 is configured so that an electronic health record system comprises an ordering system.

The reservation information includes, for example, information concerning a clinical consultation reservation, and examination reservation. The information concerning a clinical consultation reservation includes, for example, a clinical consultation day, clinical consultation time, receipt number, doctor for clinical consultation, and department for clinical consultation. The information concerning an examination reservation includes, for example, an examination day, examination time, and receipt number. The order information is, for example, information concerning an order requested by a clinical consultation doctor or the like, and is, for example, order information concerning an image examination, specimen examination, physiological examination, prescription, and medicine administration. When order information is examination order information for requesting an image examination, the examination order information includes, for example, an order number that can identify an examination, patient ID, examination type, examination region, and request source information. The order number is a number issued when examination order information is input, and is, for example, an identifier for uniquely specifying examination order information in a hospital. The examination type includes an X-ray examination, CT (Computed Tomography) examination, MR (Magnetic Resonance) examination, and RI (Radio Isotope) examination. The examination region includes, for example, abdomen, brain, and breast. The request source information includes a clinical consultation department name, the name of a doctor in charge, and the like. The information concerning an examination reservation is linked to order information.

The RIS 3 is a system that manages examination reservation information associated with a radiographic examination service. For example, the RIS 3 collects examination order information input from clinical consultation doctors in an order system included in the HIS 2 upon adding various types of setting information to the examination order information, and manages the collected information as examination reservation information. Note that the RIS 3 may add various types of setting information to examination order information by using radiation records recording various types of setting information set in the medical image diagnosis apparatus 4 in past examinations. The RIS 3 transmits an examination order to the medical image diagnosis apparatus 4 according to examination reservation information. The RIS 3 also transmits the examination execution information generated by the medical image diagnosis apparatus 4 used to execute an examination to an electronic health record system included in the HIS 2.

The medical image diagnosis apparatus 4 is an apparatus that executes an examination by, for example, imaging a patient. The medical image diagnosis apparatus 4 comprises, for example, an X-ray computed tomography apparatus, X-ray diagnosis apparatus, magnetic resonance imaging apparatus, nuclear medicine diagnosis apparatus, and ultrasonic diagnosis apparatus. The medical image diagnosis apparatus 4 executes an examination based on, for example, the examination reservation information transmitted from the RIS 3. The medical image diagnosis apparatus 4 generates examination execution information and transmits it to the RIS 3.

The medical image diagnosis apparatus 4 generates medical image data by executing an examination. The medical image data is, for example, X-ray CT image data, X-ray image data, MRI image data, nuclear medicine image data, or ultrasonic image data. The medical image diagnosis apparatus 4 generates a medical image file by converting generated medical image data into a form complying with the DICOM (Digital Imaging and Communication in Medicine) standards. A medical image file is, for example, a file in a form complying with the DICOM standards. The medical image diagnosis apparatus 4 transmits the generated medical image file to the PACS 5.

The PACS 5 is a system that manages various types of medical image files. The PACS 5 stores, for example, the medical image file transmitted from the medical image diagnosis apparatus 4. Note that the PACS 5 may store report information attached to a medical image file or report information about examinations associated with a plurality of medical image files.

The DWH 6 is a database system that collectively accumulates, for example, information generated by healthcare providers, that is, medical treatment big data. The DWH 6 is implemented by, for example, a general server apparatus. For example, as shown in FIG. 1, the DWH 6 comprises a processing circuitry 61, a memory 62, and a communication interface 63. The processing circuitry 61, the memory 62, and the communication interface 63 are communicably connected to each other via, for example, a bus.

The processing circuitry 61 is a processor that functions as the main unit of the DWH 6. The processing circuitry 61 executes programs stored in the memory 62 or the like to implement the functions corresponding to the programs. For example, a function of collecting desired information from the HIS 2, the RIS 3, the medical image diagnosis apparatus 4 and the PACS 5 and a function of causing the memory 62 to store the collected information are usable as appropriate. As a result, for example, information on electronic health records is collected from the HIS 2, examination reservation information and the like are collected from the RIS 3, and medical image files are collected from the medical image diagnosis apparatus 4 or the medical image management system 5. Further, for example, the medical information collected from the HIS 2 is stored in the memory 62 according to a preset rule. The preset rule is, for example, a sequence using event dates and times and the like associated with medical treatment events for each patient, such as outpatient consultation, surgery, image examination, specimen examination, bacteria examination, electrocardiographic measurement, vital sign measurement, medicine administration, report creation, and medical record description. The event date and time includes, for example, a date and time at which a medical treatment event has occurred or a date and time at which a medical treatment event is scheduled. The event dates and time include, for example, the execution date and time of an image examination, the execution date and time of a specimen examination, the measurement date and time of an electrocardiographic waveform, the measurement date and time of a vital sign, the date and time of administration of a medicine, the creation date and time of a report, and the date of description of a medical record. The medical information collected in this manner is stored as log information 621 in the memory 62.

The memory 62 is a storage device such as an HDD (hard disk drive), SSD (solid state drive), or integrated circuit storage device that stores various types of information. The memory 62 stores a control program and the like for causing the processing circuitry 61 to implement various functions of the function of collecting desired information and the function of causing the memory 62 to store the collected information. Note that the programs may be distributed to, for example, nonvolatile storage media and read out from the nonvolatile storage media and installed in the memory 62.

The communication interface 63 performs data communication among the medical information processing apparatus 1, the HIS 2, the RIS 3, the medical image diagnosis apparatus 4, and the PACS 5 via an intra-hospital network. Any communication standards may be used among the medical information processing apparatus 1, the HIS 2, the RIS 3, the medical image diagnosis apparatus 4, and the PACS 5. For example, it is possible to use, for example, the HL7 (Health Level 7), the DICOM, or the both of them.

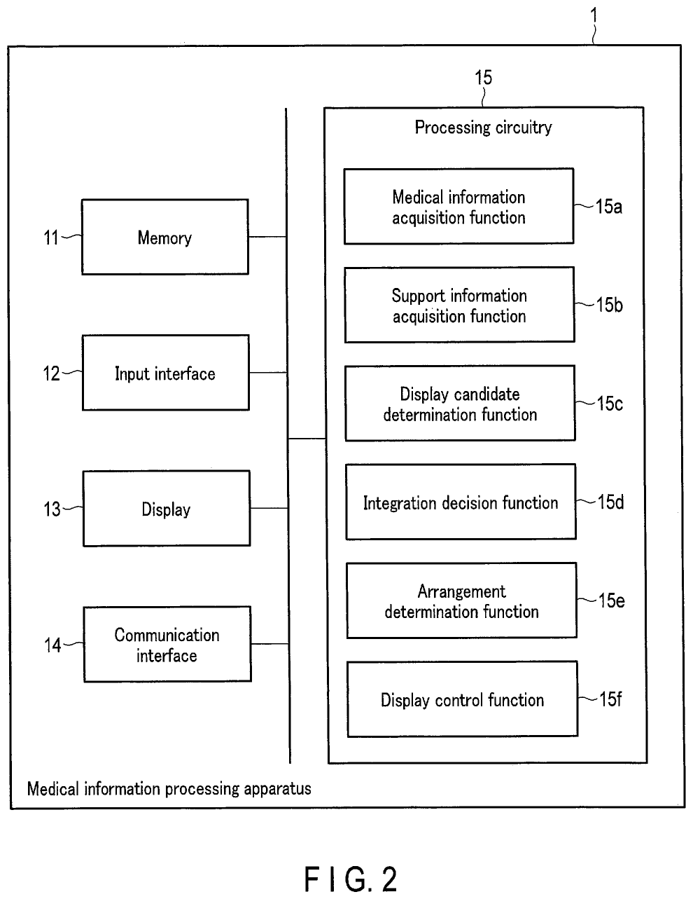

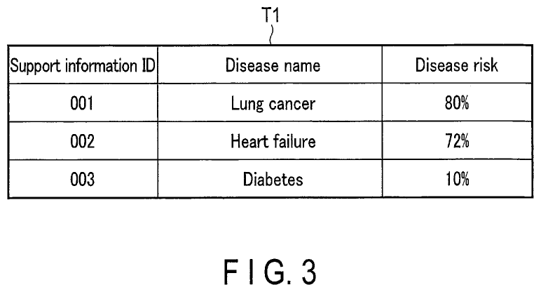

Next, the details of the medical information processing apparatus 1 according to the present embodiment will be described with reference to FIG. 2. FIG. 2 is a block diagram showing a functional configuration of the medical information processing apparatus 1 shown in FIG. 1, and FIGS. 3 to 6 are schematic diagrams describing a support information table T1, a weight table T2, and a display candidate table T3 and an integration target table T4 which are used in the medical information processing apparatus 1.

The medical information processing apparatus 1 shown in FIG. 2 comprises a memory 11, an input interface 12, a display 13, a communication interface 14, and a processing circuitry 15. The memory 11, the input interface 12, the display 13, the communication interface 14, and the processing circuitry 15 are communicably connected to one another, for example, via a bus.

Here, the memory 11 comprises a memory for recording electrical information such as a ROM (Read Only Memory), a RAM (Random Access Memory), an HDD (Hardware Disk Drive), and an image memory, and peripheral circuits such as a memory controller and a memory interface associated with the memory. The memory 11 stores, for example, various programs such as a medical information processing program of the present medical information processing apparatus, and various data such as information about an electronic health record acquired from DWH 6, various tables, data during processing, and data after processing. The information regarding the electronic health record includes, for example, patient information including a patient ID and medical information associated with the patient ID. Details of the patient information and the medical information are as described above.

Here, as shown in FIGS. 3 to 6, the various tables include the support information table T1, the weight table T2, the display candidate table T3, and the integration target table T4.

As shown in FIG. 3, a support information ID, a disease name, and a disease risk are stored in association with one another in the support information table T1. The support information ID is an identifier that can uniquely identify support information including a disease name and an index value related to the disease. The disease name is the name of a disease. However, the disease name is not necessarily limited to an official medical name as long as the disease can be identified, and a common name, a code, an abbreviation, or the like which represents the disease may be used. For example, arbitrary index values such as a disease risk, probability of hospital readmission, and probability of a side effect can be used as appropriate as an index value related to a disease. In the present specification, the index value related to the disease will be described by taking the disease risk as an example, but the present invention is not limited to this example. The disease risk is a risk calculated for each patient, and represents the probability that a patient will get a disease under a calculated condition such as 5 years later or 10 years later. However, since the disease risk may represent only the risk of contracting a disease, it is not limited to fine numerical values of 101 steps such as probability, and a classification result in which the probability is roughly classified into about 3 to 5 steps may be used. The probability of hospital readmission is the probability that a patient discharged from a hospital will be readmitted within a predetermined period, for example, within 6 weeks. The probability of a side effect is the probability that a patient will have a harmful and unintended reaction to a drug administered to the patient.

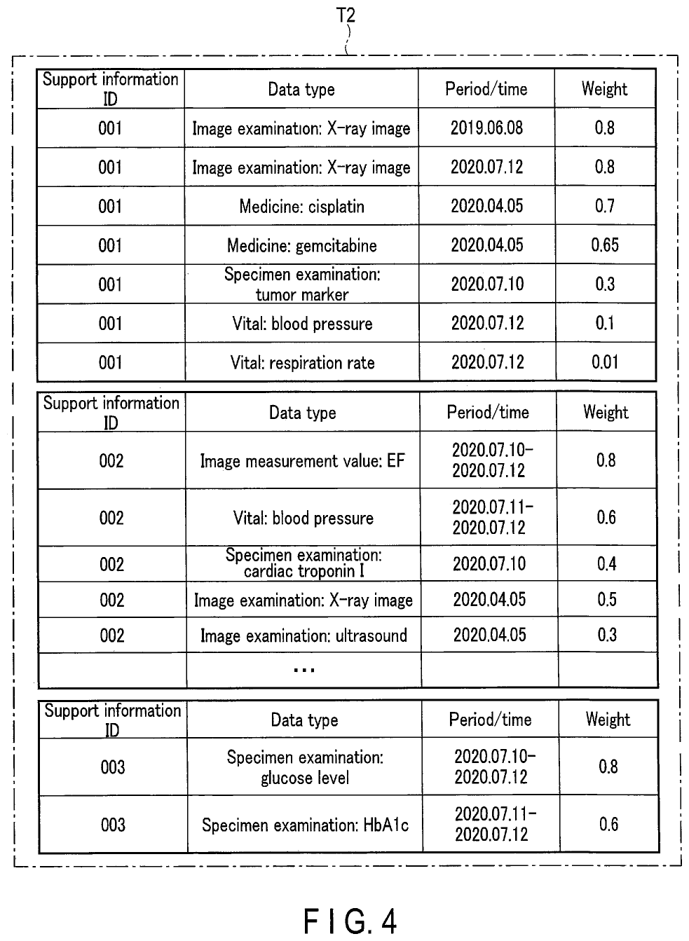

The weight table T2 is a table including medical information used for calculating the disease risk of the support information table T1. As shown in FIG. 4, a support information ID, a data type, a period/time, and a weight are written in association with one another in the weight table T2. In FIG. 4, three tables corresponding to three support information IDs are represented as the weight table T2. However, the present invention is not limited to this manner, and the tables may be aggregated into one table. Here, the medical information in the weight table T2 is information including at least the data type and the period/time among the data type, the period/time, and actual data. The support information ID is the same identifier as the support information ID in the support information table T1. The data type indicates the type of examination or the type of medicine from which actual data was acquired. The period/time is the period or date of an examination (or medication) for or on which the data indicated by the data type was acquired. Since the period is represented by two dates of a start date and an end date, the period/time may include at least the date. The actual data is data to be specified by the data type and the period/time, and represents an examination result or a medicine. The actual data is data of an examination result or medicine included in various information in the medical information in the information regarding an electronic health record, and is acquired from the DWH 6 and stored in the memory 11. The actual data is read from the memory 11 based on the patient ID, data type, and the period/time when an arrangement of medical information is determined, and is used for the determination of the arrangement. For example, in FIG. 4, the actual data regarding the data type “image examination: X-ray” is an X-ray image. The actual data regarding the data type “medicine: cisplatin” is a medication history information. The actual data regarding the data type “specimen examination: tumor marker” is examination history information. The actual data regarding the data type “vital: blood pressure” is vital sign information. The actual data regarding the data type “image measurement value: EF” is, for example, report information. The “EF” of the image measurement value is an abbreviation for ejection fraction, and represents a left ventricular ejection fraction in echocardiography. The weight is the degree to which the data indicated by the data type contributes to the calculation of a disease risk when the disease risk is calculated based on the data indicated by the data type. For example, it is assumed that the respective data of two data types are represented by A and B respectively, the data A is set to 3 points, the data B is set to 2 points, and a disease risk is calculated according to the total score of S points of both the data. In this case, from “the points of data of a weight calculation target” occupying “the total points of respective data of weight calculation targets”, the weight of the data A is calculated into 0.6(=3/(3+2)), and the weight of the data B is calculated into 0.4 (=2/(3+2)). However, the weight of the weight table T2 is not limited to the case of calculating after the fact, and for example, when a preset calculation model includes a weighting term of data, the weight in this case may be used. The weight may be renamed to another name such as the degree of service, the degree of contribution, and the degree of importance.

The display candidate table T3 is a table in which the data of rows having weights equal to or more than the threshold value in the weight table T2 are extracted for each support information ID, and the extracted data are represented in association with indexes. As shown in FIG. 5, the support information ID, the index, the data type, the period/time, and the weight are written in association with one another in the display candidate table T3. In FIG. 5, three tables corresponding to three support information IDs are represented as the display candidate table T3. However, the present invention is not limited to this style, and these tables may be aggregated into one table. The support information ID, the data type, the period/time, and the weight are the data extracted from the weight table T2. The index is a number for distinguishing the extracted data for each support information ID. The extracted data is uniquely specified by a pair of a support information ID and an index.

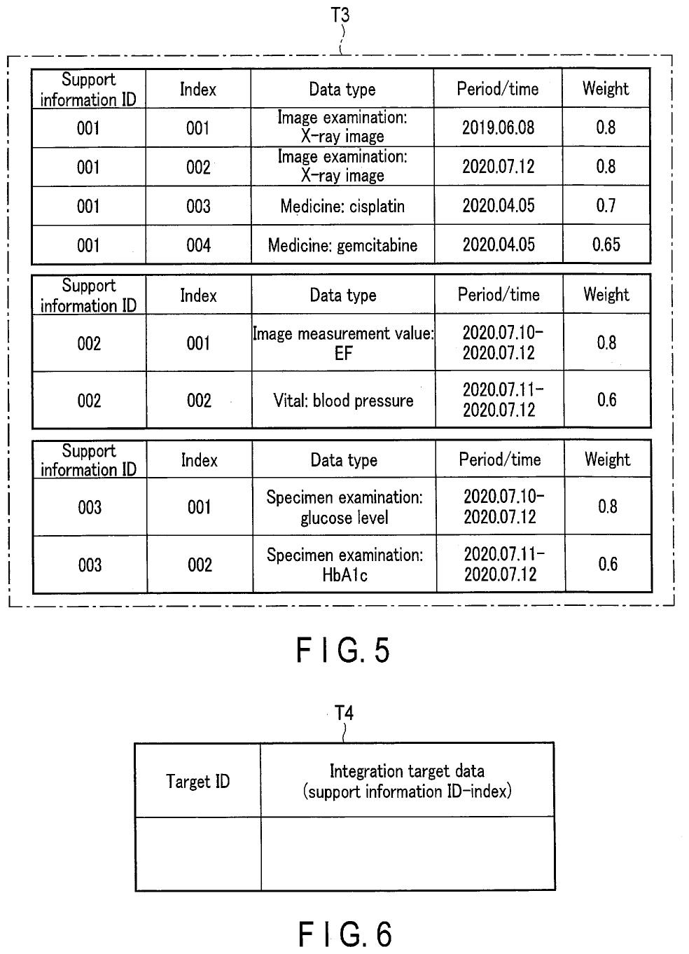

The integration target table T4 is a table for representing, as integration target data, support information IDs and indexes for specifying data of rows including a common data type among data of rows having the support information IDs adjacent to each other in the display candidate table T3. As shown in FIG. 6, a corresponding ID and integration target data are written in association with each other in the integration target table T4. The corresponding ID is an identifier for uniquely specifying the integration target data. The corresponding ID may be renamed to another name such as an integration ID or a common ID.

Returning to FIG. 2, the input interface 12 is implemented by a track ball, a switch button, a mouse, a keyboard, a touch pad (or track pad) for performing an input operation by touching an operation surface, a touch panel display (or touch screen) equipped with a display screen and a touch pad integrated with each other, or the like, which inputs various instructions, commands, information, selections, and settings from an operator (user) to the main body of the medical information processing apparatus. The input interface 12 is connected to the processing circuitry 15, converts an input operation received from the user into an electric signal, and outputs the electric signal to the processing circuitry 15. In this case, the input interface 12 may cause the display 13 to display a user interface (GUI: Graphical User Interface) through which the user inputs various commands by a physical operating component such as a mouse or a keyboard. In the present specification, the input interface 12 is not limited to only a component including a physical operating component. For example, an example of the input interface 12 comprises an electric signal processing circuitry that receives an electric signal corresponding to an input operation from an external input device provided separately from the device and outputs the electric signal to the processing circuitry 15. In the following description, “an operation of the input interface 12 by the user” is also referred to as “a user's operation”.

The display 13 comprises a display main body that displays a medical image and the like, an internal circuit that supplies a signal for display to the display main body, and a peripheral circuit including a connector and a cable that connect the display and the internal circuit. The display 13 can display arbitrary data such as patient information, health status data, and health status monitoring protocol as appropriate. The display 13 is an example of a display unit.

The communication interface 14 is a circuit for connecting the medical information processing apparatus 1 to a network and communicating with other devices. For example, a network interface card (NIC) can be used as the communication interface 14. In the following description, the description that the communication interface 14 intervenes in the communication between the medical information processing apparatus 1 and the other devices will be omitted.

The processing circuitry 15 reads out the medical information processing program stored in the memory 11 based on a command input by the user via the input interface 12, and controls the medical information processing apparatus 1 according to the read-out program. For example, the processing circuitry 15 is a processor that implements each function of the medical information processing apparatus 1 according to the medical information processing program read out from the memory 11. Examples of the functions include a medical information acquisition function 15a, a support information acquisition function 15b, a display candidate determination function 15c, an integration decision function 15d, an arrangement determination function 15e, a display control function 15f, and the like. Note that the respective functions may be implemented while distributed to a plurality of processors as appropriate. Alternatively, the respective functions or some of the respective functions may be caused to be executed by another device as appropriate. For example, the medical information acquisition function 15a, the support information acquisition function 15b, and the display control function 15f out of the respective functions may be caused to be executed by another device (not shown). In other words, the medical information acquisition function 15a, the support information acquisition function 15b, and the display control function 15f are optional additional items that are not necessarily needed to be included in the medical information processing apparatus 1, and may be omitted from the medical information processing apparatus 1.

Next, the medical information acquisition function 15a, the support information acquisition function 15b, the display candidate determination function 15c, the integration decision function 15d, the arrangement determination function 15e, and the display control function 15f as the respective functions will be described in order. However, the allotment of the respective functions described below is for convenience's sake, and can be changed as appropriate. This is because even if the processing to be allotted to a function is allotted to another function, there is no change in that the processing circuitry 15 still executes the processing. For example, the support information acquisition function 15b, the display candidate determination function 15c, the integration decision function 15d, and the arrangement determination function 15e may display data during processing or after processing on the display 13 by executing the function of the display control function 15f as appropriate. Note that it is true of embodiments and modifications described below that the allotment of the respective functions can be changed.

The medical information acquisition function 15a acquires a plurality of medical information pieces associated with the patient ID of a target patient from the DWH 6. The medical information acquisition function 15a is an example of a first acquisition unit.

The support information acquisition function 15b acquires support information including a disease risk calculated from the acquired plurality of medical information pieces based on a calculation model for calculating a disease risk from a plurality of medical information pieces, and calculates a weight for each of the acquired plurality of medical information pieces. The calculation model is a model for calculating an index value (for example, a disease risk) associated with a disease by summing up points obtained by individually scoring a plurality of medical information pieces. Note that the calculation model described above includes a model for individually scoring patient information in addition to scoring medical information, and summing up both the scores (points) to calculate an index value associated with a disease. For example, a coronary artery disease onset prediction model based on Suita Score in the 2017 edition of the Japan Atherosclerosis Society's Arteriosclerosis Prevention Guidelines, and the like are known as this type of calculation model. This coronary artery disease onset prediction model is such that the points of eight factors such as (1) age, (2) sex, (3) smoking, (4) blood pressure, (5) HDL-C (mg/dl), (6) LDL-C (mg/dl), (7) resistance to dysfunction, (8) family history of early-onset coronary artery disease are totaled, and the total score is classified into a low-risk, a medium-risk, or a high-risk. In this example, the factors (1) to (3) and (8) correspond to the patient information, and the factors (4) to (7) correspond to the medical information. Further, the support information acquisition function 15b calculates each of disease risks from the acquired plurality of medical information pieces based on the calculation model, and acquires a plurality of support information pieces each of which individually includes each of the disease risks. Further, the support information acquisition function 15b acquires the plurality of support information pieces, and calculates the ratio of the score of each of the acquired plurality of medical information pieces to the total score of the plurality of medical information pieces as the weight of each of the plurality of medical information pieces. The support information acquisition function 15b is an example of a second acquisition unit.

The display candidate determination function 15c determines each medical information as a display candidate based on a plurality of support information pieces each of which individually includes each of a plurality of disease risks, and the weight of each of a plurality of medical information pieces, which is related to each of the plurality of disease risks. Here, the display candidate determination function 15c may determine each medical information serving as a display candidate by using the acquired plurality of support information pieces and the respective weights. For example, the display candidate determination function 15c may determine medical information having a weight equal to or more than a threshold value as a display candidate by comparing each of the related plurality of medical information pieces with the threshold value for each support information. The display candidate determination function 15c is an example of a determination unit.

Based on respective medical information pieces which have been determined to be display candidates, and the plurality of support information pieces, the integration decision function 15d decides whether or not to integrate the respective medical information pieces. Each of the plurality of medical information pieces may include a data type and a date. In this case, the integration decision function 15d may make decision so as to integrate respective medical information pieces containing the same data type and dates within the same period among a plurality of medical information pieces used to calculate adjacent disease risks when a plurality of disease risks are arranged in order. The integration decision function 15d is an example of a decision unit.

As a result of the decision, the arrangement determination function 15e determines the arrangement of medical information pieces to be integrated and medical information pieces to be non-integrated based on a plurality of support information pieces. For example, the arrangement of the medical information pieces to be integrated may be determined so as to be related to the plurality of support information pieces, but the arrangement of the medical information pieces to be non-integrated may be determined so as to be related to a single support information piece without being related to the plurality of support information pieces. Specifically, for example, the arrangement may be determined so that the arrangement direction of the medical information pieces to be non-integrated and the arrangement direction of the medical information pieces to be integrated are orthogonal to each other. Specifically, for example, the arrangement may be determined so that the medical information pieces to be non-integrated are arranged along the direction of a row or column in association with each support information piece, and the medical information pieces to be integrated are arranged along the direction of a column or row orthogonal to the former direction in association with a plurality of pieces of support information.

Further, with respect to each of the medical information pieces to be non-integrated, the display range thereof may be calculated based on each disease risk. For example, with respect to first medical information related to a lung cancer risk (80%), second medical information related to a heart failure risk (72%), and third medical information related to a diabetes risk (10%), the display ranges thereof may be calculated as follows. For example, the display range of the first medical information may be set to 80/(80+72+10)=0.5 (approximate value) so as to calculate the ratio of “the disease risk related to the first medical information” to “the total value of the disease risks related to the first to third medical information”. Similarly, the display range of the second medical information may be set to 72/(80+72+10)=0.4 (approximate value) so as to calculate the ratio of “the disease risk related to the second medical information” to “the total value of the disease risks related to the first to third medical information”.

The display range of the third medical information may be set to 10/(80+72+10)=0.1 (approximate value) so as to calculate the ratio of “the disease risk related to the third medical information” to “the total value of the disease risks related to the first to third medical information”. In other words, the display range may be calculated so as to have a size proportional to the disease risk. The calculated display range can be used as the height of each row, for example, when each medical information is arranged in each row. Similarly, the calculated display range can be used as the width of each column, for example, when each medical information is arranged in each column.

When each medical information is arranged in each row, each medical information may be arranged in descending order of weight in each row. Note that the descending order is the order from a larger number to a smaller number. Similarly, when each medical information is arranged in each column, each medical information may be arranged in descending order of weight in each column. Here, even if medical information has a weight smaller than the threshold value, the medical information may be arranged so as to be displayed in descending order of the weight by scrolling. As a result, for example, when medical information having a weight equal to or more than the threshold value is a display candidate, medical information having a weight smaller than the threshold value is not displayed on the initial screen because it is not a display candidate, but it is displayed in descending order of weight by scrolling. The arrangement determination function 15e is an example of an arrangement determination unit.

The display control function 15f controls the display 13 so as to display a plurality of support information pieces and medical information pieces as display candidates based on the determined arrangement. The display control function 15f is an example of a display control unit.

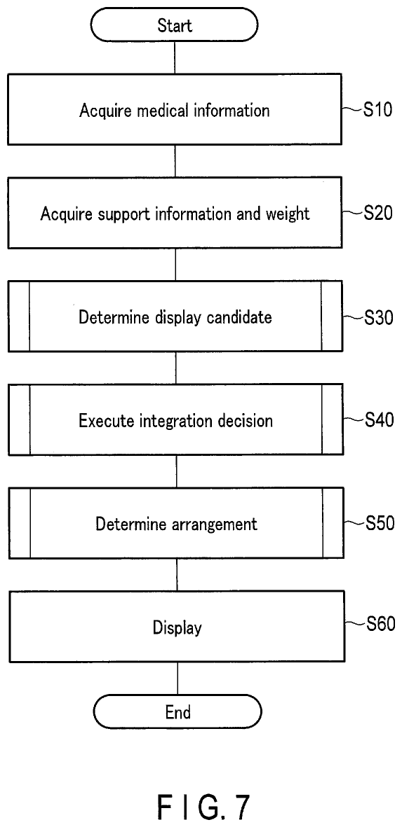

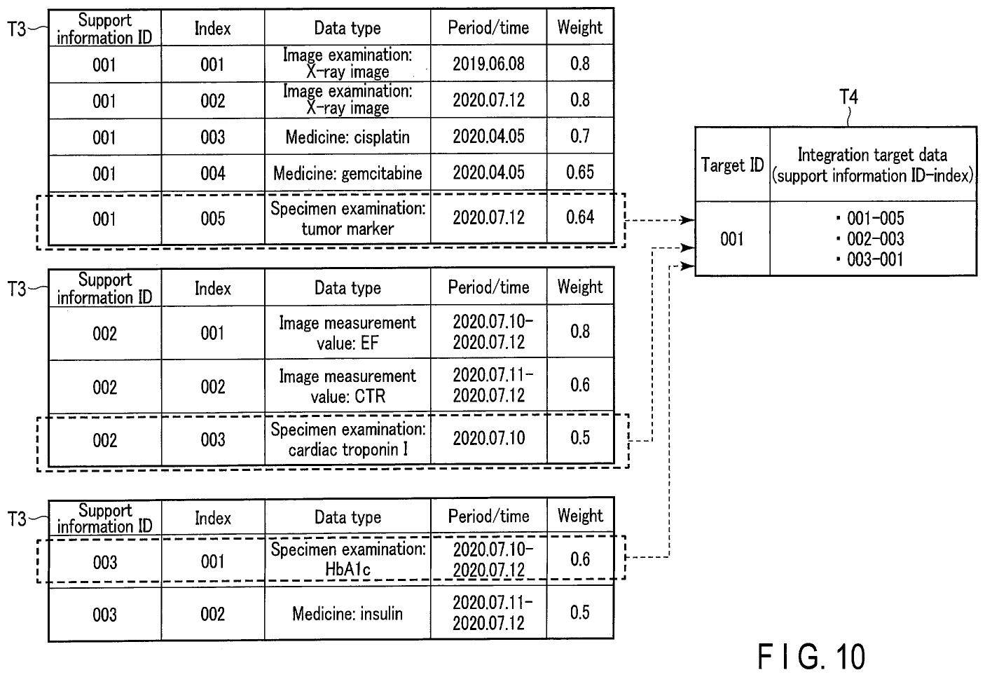

Next, an operation of the medical information processing system including the medical information processing apparatus configured as described above will be described by using flowcharts of FIGS. 7 to 9 and 11 and schematic views of FIGS. 10 and 12 to 14.

It is assumed that the DWH 6 has stored patient information and patient's medical information in the memory 62 at present. Step ST10 is started in this state.

In step ST10, the processing circuitry 15 of the medical information processing apparatus 1 acquires patient information and a plurality of medical information pieces from the DWH 6 based on the patient ID of a target patient, and stores the acquired patient information and the plurality of medical information pieces in the memory 11. The medical information stored in the memory 11 includes a data type, a period/time, and actual data.

After step ST10, in step ST20, based on a plurality of medical information pieces and a calculation model for calculating a disease risk of a disease having a disease name, the processing circuitry 15 acquires support information containing disease risks calculated from the acquired plurality of medical information pieces, and calculates the weight of each of the acquired plurality of medical information pieces. For example, it is assumed that the calculation model is a model for calculating a disease risk by summing up the points obtained by individually scoring a plurality of medical information pieces. At this time, the processing circuitry 15 calculates each of the disease risks from the plurality of medical information pieces in the memory 11 based on the calculation model, and acquires a plurality of support information pieces each of which individually contains each of the disease risks. Further, the processing circuitry 15 writes the acquired support information (disease name, disease risk) into the support information table T1 while associating the acquired support information with the support information ID. Further, for example, the processing circuitry 15 acquires the support information, and calculates, as the weight of each of the medical information pieces, the ratio of the score of each of the plurality of medical information pieces (a plurality of actual data specified by a plurality of data types and period/time) in the memory 11 to the total score. Thereafter, the processing circuitry 15 writes the calculated weight into the weight table T2 while associating the calculated weight with the medical information and the support information ID used for the calculation. However, the actual data out of the data type, the period/time, and the actual data included in the medical information used for calculation is not written in the weight table T2, but the data type and the period/time for specifying the actual data are written in the weight table T2.

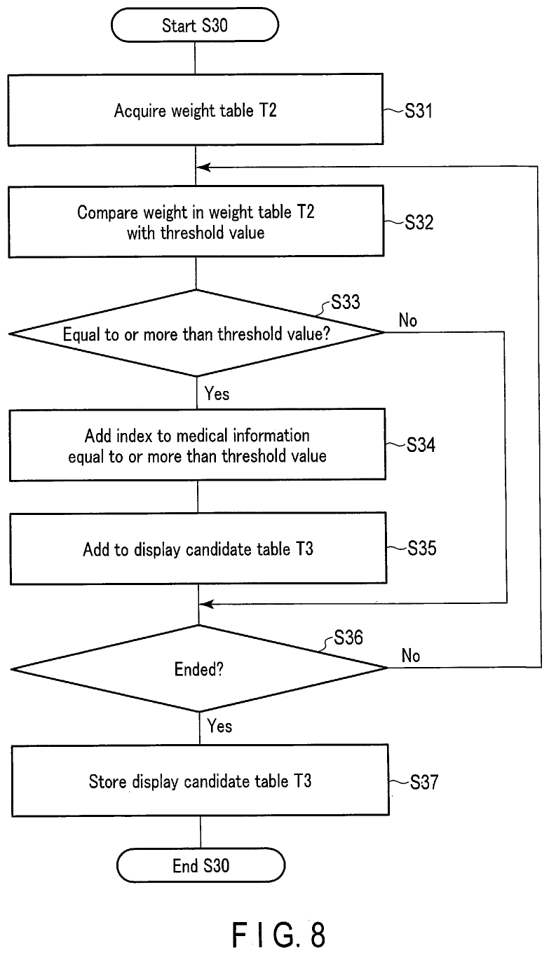

After step ST20, in step ST30, the processing circuitry 15 determines each medical information serving as a display candidate based on a plurality of support information pieces each individually including each of a plurality of disease risks and the weight of each of a plurality of medical information pieces related to each of a plurality of disease risks. Such step ST30 is executed, for example, by steps S31 to S37 shown in FIG. 8.

In other words, the processing circuitry 15 acquires the weight table T2 from the memory 11 (step S31). The processing circuitry 15 compares the weight in the weight table T2 with a threshold value (step S32) to determine whether or not the weight is equal to or more than the threshold value (step S33). When the weight is equal to or more than the threshold value, the processing circuitry 15 adds an index to medical information having a weight equal to or more than the threshold value (step S34), adds the index-added medical information to the display candidate table T3 (step S35), and then shifts to step S36. In other words, the medical information having a weight equal to or more than the threshold value is written in the display candidate table T3 as a display candidate. If the result of the determination in step S33 is negative, the processing shifts to step S36.

In step S36, the processing circuitry 15 determines whether or not the processing of step S32 and subsequent steps thereto is completed for the data in all the rows in the weight table T2. If the determination is negative, the processing circuitry 15 returns to step S32 to execute the processing on data in unprocessed rows. Further, when the processing is completed as a result of the determination in step S36, the processing circuitry 15 stores the display candidate table T3 in the memory 11 and ends the step S30.

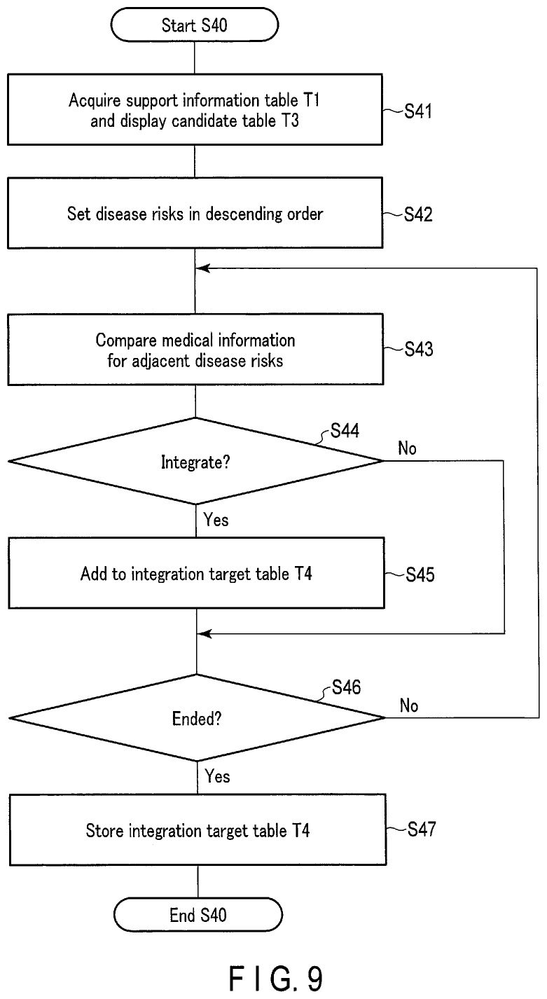

Returning to FIG. 7, after step ST30, in step ST40, based on the respective medical information pieces serving as the display candidates and the plurality of support information pieces, the processing circuitry 15 determines whether the respective medical information pieces should be integrated or not. For example, the determination may be made so as to integrate respective medical information pieces containing the same data type and dates within the same period among the plurality of medical information pieces used for calculating adjacent disease risks when a plurality of disease risks are arranged in order. Such step ST40 is executed, for example, by steps S41 to S47 shown in FIG. 9.

In other words, the processing circuitry 15 acquires the support information table T1 and the display candidate table T3 from the memory 11 (step S41). The processing circuitry 15 rearranges the data of each row in the support information table T1 so that the disease risks are arranged in descending order (step S42). The processing circuitry 15 compares a plurality of medical information pieces for adjacent disease risks (step S43), and determines whether or not to integrate a plurality of medical information pieces according to whether they include the same data type and dates within the same period (step S44). When the plurality of medical information pieces are integrated, for example, as shown in FIG. 10, the processing circuitry 15 adds corresponding IDs to integration target data (support information ID and indexes) for specifying integration targets, adds them to the integration target table T4 (step S45), and shifts to step S46. In other words, medical information pieces having common data type and examination period are written as integration targets in the integration target table T4. If the result of the determination in step S44 is negative, the processing shifts to step S46. In FIG. 10, “CTR” of the image measurement value is an abbreviation for cardio-thoracic ratio, and represents the ratio of the width of the heart to the width of the thorax in the chest X-ray. The “X-ray” may be referred to as an “X-ray image” or an “X-ray examination”.

In step S46, the processing circuitry 15 determines whether or not the processing of step S43 and subsequent steps thereto has been completed for the data in all the rows in the display target table T3, and if the determination is negative, the processing circuitry 15 returns to step S43 to execute the processing on the data of unprocessed rows. Further, when the processing is completed as a result of the determination in step S46, the processing circuitry 15 stores the integration target table T4 in the memory 11 (step S47), and ends the step S40.

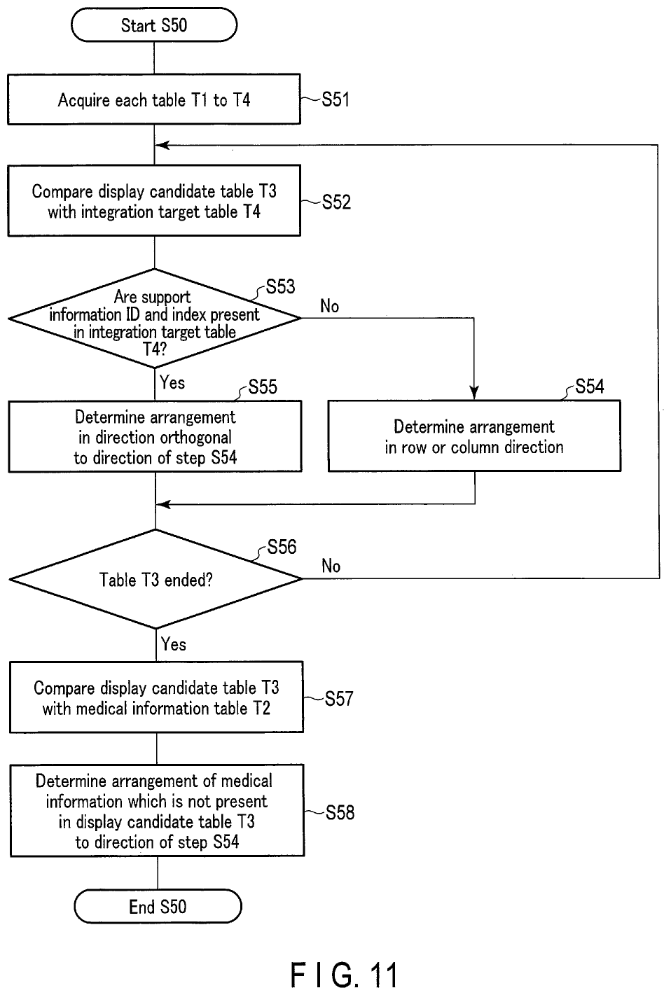

Returning to FIG. 7, after step ST40, in step ST50, as a result of the determination, the processing circuitry 15 determines the arrangement of the medical information pieces to be integrated and the arrangement of the medical information pieces to be non-integrated based on the plurality of support information pieces. For example, the processing circuitry 15 may determine the arrangement so that the arrangement direction of the medical information pieces to be non-integrated and the arrangement direction of the medical information pieces to be integrated are orthogonal to each other. Specifically, for example, the processing circuitry 15 may determine the arrangement so that the medical information pieces to be non-integrated are arranged along the row or column direction in association with each support information, and also determine the arrangement so that the medical information pieces to be integrated are arranged along the column or row direction orthogonal to the former direction in association with a plurality of support information pieces. Such step ST50 is executed, for example, by steps S51 to S58 shown in FIG. 11.

In other words, the processing circuitry 15 acquires the support information table T1, the weight table T2, the display candidate table T3, and the integration target table T4 from the memory 11 (step S51). The processing circuitry 15 compares the display candidate table T3 with the integration target table T4 (step S52), and determines whether the support information ID and the index of each row in the display candidate table T3 are present in the integration target table T4 (Step S53). When they are not present in the integration target table T4, the processing circuitry 15 determines the arrangement so that the medical information pieces to be non-integrated are arranged along the row or column direction in association with disease names and disease risks (step S54). Which one of the row direction and the column direction should be set as the arrangement direction may be determined, for example, based on initial setting or according to the presence or absence of data suitable for display in the row direction. Further, when the support information ID and the index are present in the integration target table T4 as a result of the determination in step S53, the processing circuitry 15 determines the arrangement so that the medical information pieces to be integrated are arranged in association with disease names and disease risks along a direction orthogonal to the direction used in step S54 (step S55).

After the end of step S54 or S55, the processing circuitry 15 determines whether the processing of step S52 and subsequent steps thereto has been completed for the data of all the rows in the display target table T3 (step S56). If the result of the determination in step S56 is negative, the processing circuitry 15 returns to step S52 to execute the processing on the data of the unprocessed rows. Further, when the processing is completed as a result of the determination in step S56, the processing circuitry 15 compares the display candidate table T3 with the medical information table T2 (step S57). As a result of the comparison, the processing circuitry 15 determines the direction used in step S54 for the arrangement of the medical treatment direction that is not in the display candidate table T3 (step S58), and ends the processing in step S50.

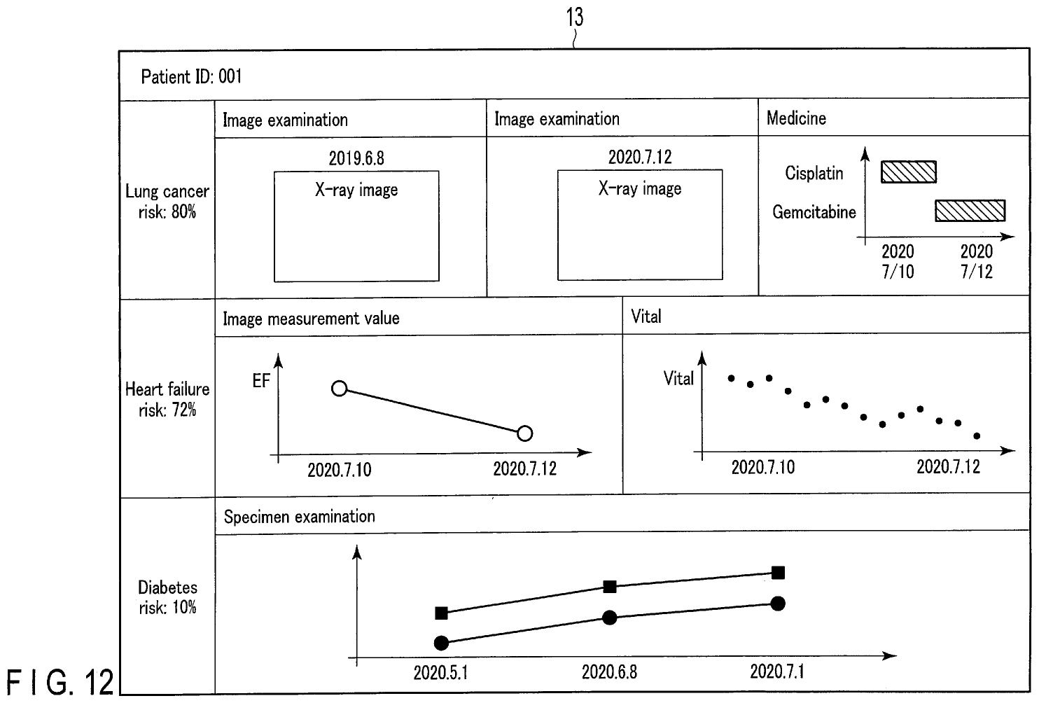

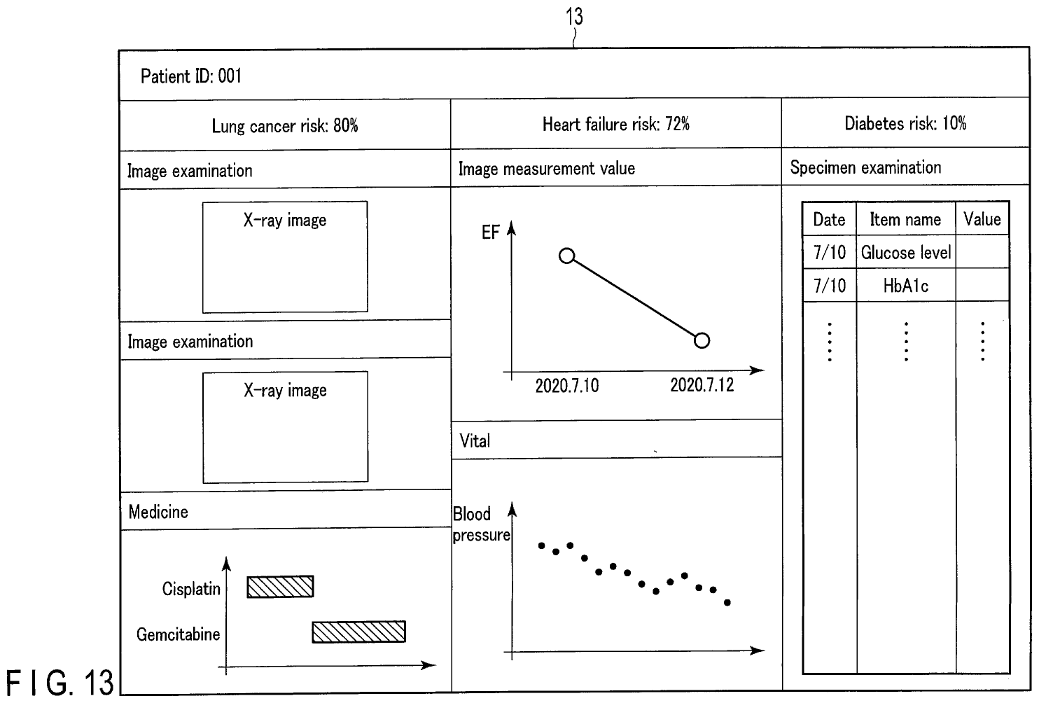

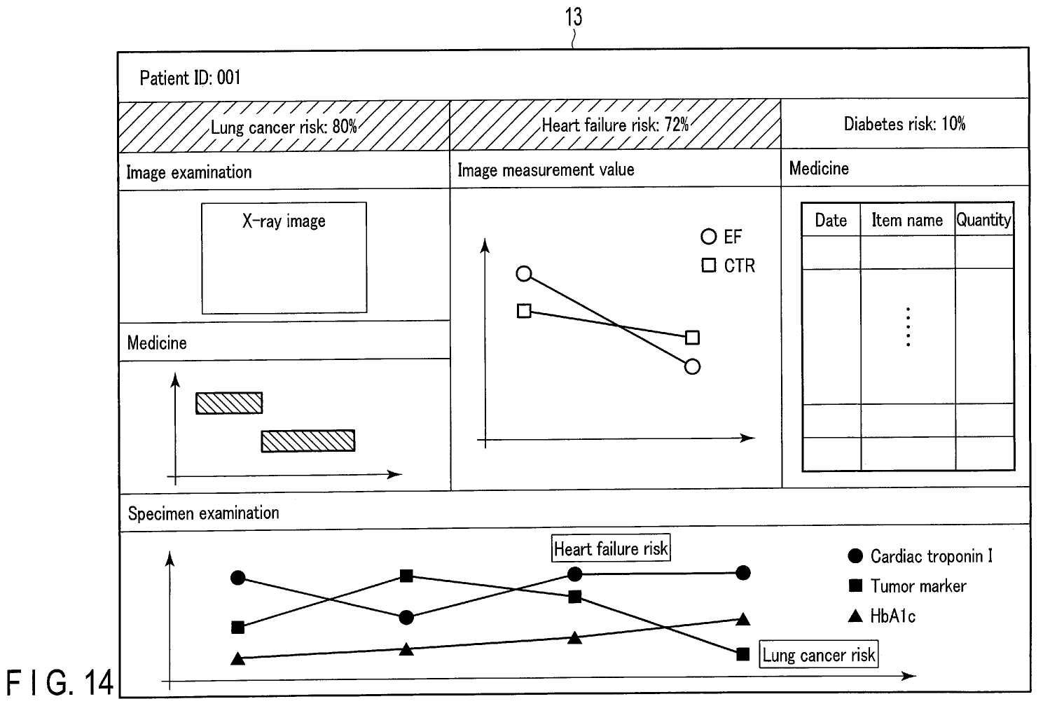

Returning to FIG. 7, after step ST50, in step S60, the processing circuitry 15 controls the display 13 to display a plurality of support information pieces and medical information as a display candidate based on the determined arrangement. At this time, the processing circuitry 15 may read out a plurality of actual data of a plurality of medical information pieces from the memory 11 based on the data types and the period/time of the plurality of medical information pieces associated with the patient ID, and cause the display 13 to display a screen on which a plurality of support information pieces and a plurality of actual data are arranged. Alternatively, when determining the arrangement in step ST50, the processing circuitry 15 may read out a plurality of actual data of the plurality of medical information pieces from the memory 11 based on the data type and the period/time of the plurality of medical information pieces related to the patient ID, and determine the arrangement by using a plurality of support information pieces and a plurality of actual data. In any case, the display 13 displays a plurality of support information pieces and a plurality of medical information based on the determined arrangement, for example, as shown in any of FIGS. 12 to 14. Note that FIGS. 12 and 13 correspond to a case where there is no medical information piece to be integrated between adjacent disease risks, and FIG. 14 corresponds to a case where there are medical information pieces to be integrated and medical information pieces to be non-integrated between adjacent disease risks.

Giving some supplemental description, FIG. 12 shows a screen on the display 13 when the arrangement is determined so that the medical information pieces to be non-integrated are arranged side by side along the row direction in association with disease names and disease risks. In FIG. 12, the medical information pieces to be non-integrated are displayed in display regions which are individually associated with respective disease names and disease risks. The medical information pieces (data type, period/time) to be displayed on the screen are ones written in the display candidate table T3. Medical information which is not present in the display candidate table T3 is scrolled and displayed from medical information on the screen according to a user's operation. Therefore, the directions used in steps S54 and S58 are set to be the same.

FIG. 13 shows a screen on the display 13 when the arrangement is determined so that the medical information pieces to be non-integrated are arranged side by side along the column direction unlike FIG. 12. In FIG. 13, the medical information pieces to be non-integrated are displayed in display regions which are individually associated with respective disease names and disease risks.

FIG. 14 shows a screen on the display 13 when the arrangement is determined so that the medical information pieces to be non-integrated are arranged side by side along the column direction while the medical information pieces to be integrated are arranged side by side along the row direction (the direction orthogonal to the column direction) in association with the disease names and the disease risks. In other words, in FIG. 14, the medical information pieces to be non-integrated are displayed in the display regions which are individually associated with respective disease names and disease risks. The medical information pieces to be integrated are displayed in a display region associated with a plurality of disease names and disease risks.

In any of FIGS. 12 to 14, individual related medical information pieces can be presented to the user for a plurality of disease names and disease risks within a limited display range.

As described above, according to the first embodiment, each medical information piece as a display candidate is determined based on a plurality of support information pieces which individually includes index values (for example, disease risks) relating to a plurality of diseases, and a weight of each of a plurality of medical information pieces associated with each of the index values relating to the plurality of diseases. Further, it is determined whether or not to integrate respective medical information pieces based on each medical information piece as a display candidate and a plurality of support information pieces. As a result of the determination, the arrangements of the medical information pieces to be integrated and the medical information pieces to be non-integrated are determined based on a plurality of support information.

Therefore, respective related medical information pieces can be presented to a user for a plurality of support information regardless of the period of the medical information within a limited display range. Giving some supplemental description, when there are a plurality of support information and respective related medical information, the medical information for determining the validity of the support information can be presented to the user within a limited display range. For example, even if there is medical information which has a large weight and deviates from the time axis of the same period, the medical information is arranged as medical information to be non-integrated, and thus it can be presented to the user.

Further, even in comparison with a case where a plurality of support information pieces are sequentially (individually) confirmed, it is not necessary to switch and display each support information piece, so that it is possible to efficiently grasp the validity of the support information.

Further, according to the first embodiment, each of the plurality of medical information pieces may include a data type and a date. In addition, the determination may be made so as to integrate respective medical information pieces containing the same data type and dates within the same period out of a plurality of medical information pieces used to calculate adjacent index values when index values relating to a plurality of diseases are arranged in order. In this case, medical information pieces related to index values for a plurality of diseases can be integrated and presented.

Further, according to the first embodiment, the arrangement may be determined so that the medical information pieces to be non-integrated are arranged side by side along the row or column direction in association of each support information piece, and the arrangement may be determined so that the medical information pieces to be integrated are arranged side by side in the column or row direction orthogonal to the former direction in association with a plurality of support information pieces. In this case, the arrangement can be determined so that the medical information pieces to be non-integrated are arranged side by side in association with a single support information piece (the index value related to a single disease), and the medical information pieces to be integrated are arranged side by side in association with a plurality of support information pieces, so that it is possible to determine the arrangement corresponding to the relation between the medical information and the support information.

Further, according to the first embodiment, a plurality of medical information for a target patient may be acquired. Further, based on a calculation model for summing up points obtained by individually scoring a plurality of medical information pieces to calculate index values related to diseases, each of the index values related to the disease may be calculated from the acquired plurality of medical information pieces to acquire a plurality of support information pieces which individually include each of the index values related to the diseases, and the ratio of the points of each of the acquired plurality of medical information pieces to the total points may be calculated as the weight of each of the medical information pieces. Further, respective medical information pieces as display candidates may be determined by using the acquired plurality of support information and the respective calculated weights. In this case, the disease risk and the weight can be easily calculated based on the calculation model.

Further, according to the first embodiment, the display unit (display 13) may be controlled so as to display a plurality of support information and medical information as a display candidate based on the determined arrangement. In this case, based on the determined arrangement, the display unit can display a screen in which the support information and the medical information are arranged.

The first embodiment as described above may be modified as shown in the following modification.

This modification is configured so that instead of the calculation based on the calculation model, index values related to diseases and respective weights of the plurality of medical information pieces are stored in the memory 11 in advance in association with the plurality of medical information pieces. The memory 11 is an example of a storage unit.

Along with this, the support information acquisition function 15b of the processing circuitry 15 refers to the memory 11 based on the acquired plurality of medical information to acquire a plurality of support information individually including index values (for example, disease risks) related to a plurality of diseases and respective weights of a plurality of medical information related to the respective index values related to a plurality of diseases. The support information acquisition function 15b is another example of the second acquisition unit.

The other configurations are the same as in the first embodiment.

In such a modification, the operation of step S20 is mainly different from that of the first embodiment. For example, step S10 is executed in the same manner as described above, and the processing circuitry 15 acquires a plurality of medical information in a target patient.

Further, in step S20, the processing circuitry 15 refers to the memory 11 based on the acquired plurality of medical information to acquire a plurality of support information which individually include the plurality of disease risks, and a weight of each of a plurality of medical information which is related to each of the plurality of disease risks.

Thereafter, step S30 is executed in the same manner as described above, and the processing circuitry 15 determines respective medical information pieces as display candidates by using the acquired plurality of support information and the respective weights.

Steps S40 to S60 are executed in the same manner as described above.

According to the above-mentioned modification, a plurality of support information which individually includes index values (for example, disease risks) related to the plurality of diseases, and respective weights of a plurality of medical information related to the respective index values related to the plurality of diseases can be acquired by using no calculation model. Further, according to this modification, the same action and effect as those of the first embodiment can be obtained except for the action and effect related to the calculation model.

### Second Embodiment

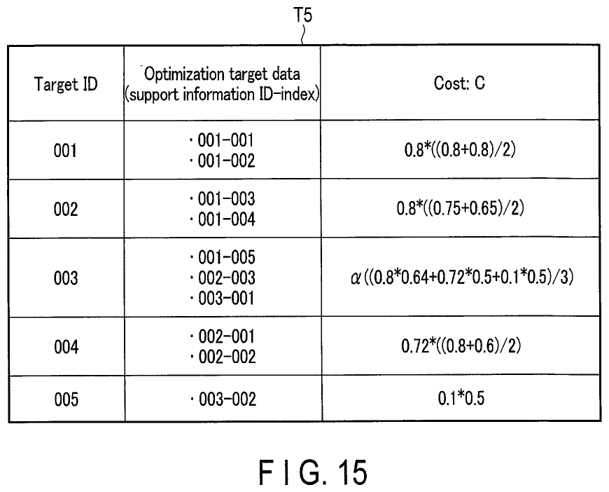

Next, a medical information processing apparatus according to a second embodiment will be described with reference to FIG. 15. In the following description, substantially the same elements as those in the above-mentioned figures are designated by the same reference signs, detailed description thereof will be omitted, and different elements will be mainly described.

The second embodiment is configured so that the size of a display region for medical information pieces to be integrated and the size of a display region for medical information pieces to be non-integrated are adjusted unlike the first embodiment in which the arrangement direction is changed between the medical information pieces to be integrated and the medical information pieces to be non-integrated.

Along with this, a memory 11 stores an optimization target table T5 as shown in FIG. 15 instead of the above-mentioned integration target table T4. A target ID, optimization target data (support information ID-index), and a cost C are written in association with one another in the optimization target table T5. The optimization target data includes at least one pair of a support information ID and an index that specify the data of rows to be displayed in the same display region among the data of the rows in the display candidate table T3. The cost C is an index based on an index value (for example, disease risk) and a weight related to a disease in the data in the row specified by the optimization target data, and is a value indicating the magnitude proportional to the index value and the weight. The cost C is calculated, for example, by using the weight and the disease risk in the row specified by the optimization target data. In FIG. 15, when the target ID has a common support information ID in the optimization target data as shown in the rows of target IDs 001, 002, and 004, the cost C is calculated by multiplying the average value of the weights by the disease risk. However, the present invention is not limited to this manner, and the cost C may be calculated by a mathematically equivalent method such as calculation of a weighted average value when respective weights are multiplied by disease risks. Further, in FIG. 15, when the target ID has different support information IDs in the optimization target data as shown in the row of target ID 003, the cost C is calculated by calculating a weighted average value when respective weights are multiplied by disease risks, and multiplying the weighted average value by an arbitrary coefficient α (0<α≤1). The coefficient α is a value for adjusting the magnitude of the cost of common data. Further, when the target ID has one optimization target data as shown in the row of target ID 0005 in FIG. 15, the cost C is calculated by multiplying a weight by a disease risk. Note that the term “cost” may be changed to another name as appropriate. The target ID is an identifier for uniquely identifying the optimization target data. The target ID may be renamed to another name such as an adjustment ID or a display region ID. Since “the optimization target table” does not necessarily mean “table of targets to be made most appropriate”, it may be renamed to a name of “target table” from which “most” is removed like “suiting target table” or “adjustment target table”.

Further, an integration decision function 15d of a processing circuitry 15 creates and updates the optimization target table T5 based on a support information table T1 and a display candidate table T3 in the memory 11, and stores the optimization target table T5 into the memory 11.

The arrangement determination function 15e of the processing circuitry 15 determines the arrangement by adjusting the size of the display region of the medical information to be non-integrated and the size of the display region of the medical information to be integrated based on the disease risks and the weights. For example, the arrangement determination function 15e adjusts the size of the display region of the medical information based on the cost C (the index based on the disease risk and the weight) in the optimization target table T5. Here, the arrangement determination function 15e may adjust the size of the display region so that the order of the sizes is proportional to the disease risk and the weight. For example, the arrangement determination function 15e may adjust the size of the display region of medical information so that the size of the display region is matched with the order of the size of the cost C in the optimization target table T5.

The other configurations are the same as in the first embodiment. The second embodiment may be applied to the modification of the first embodiment.

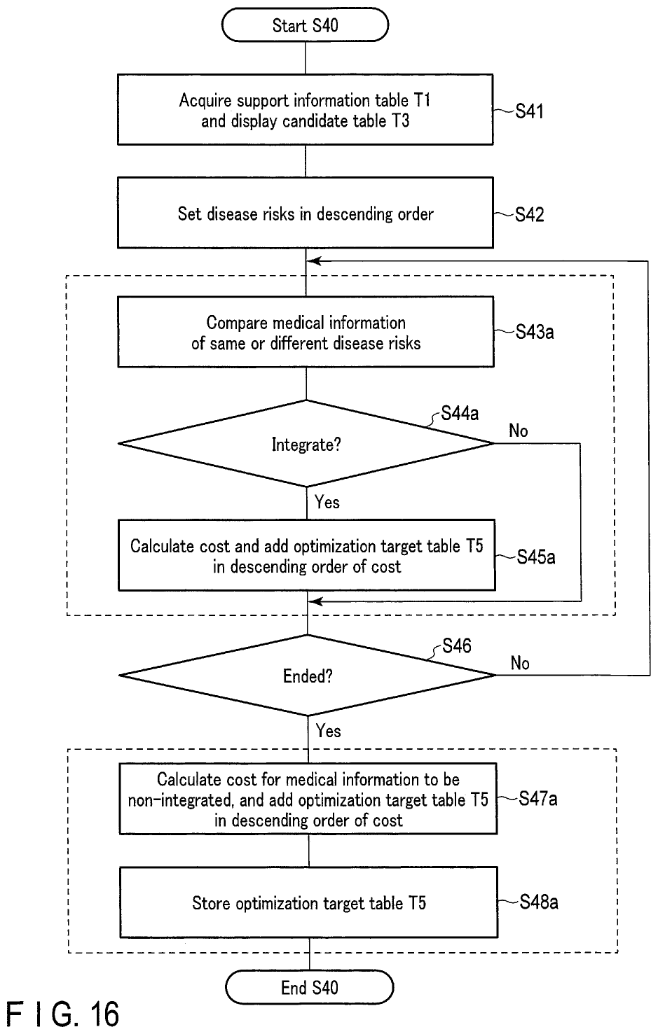

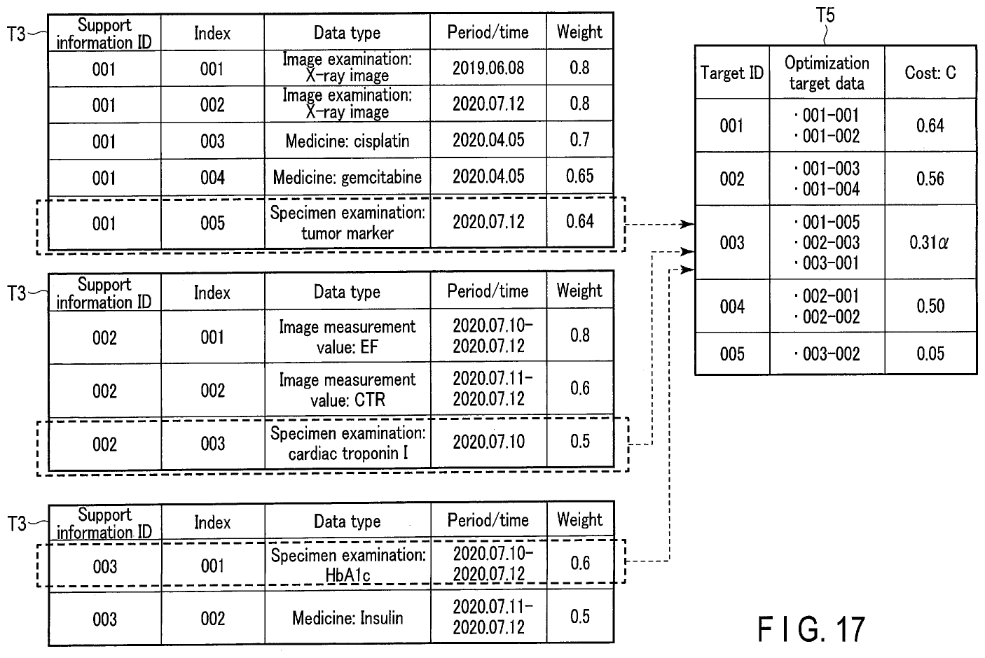

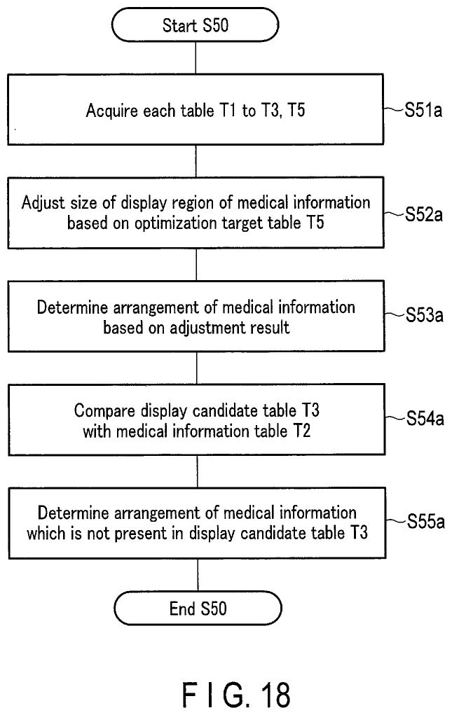

Next, the operation of the thus-configured medical information processing apparatus will be described with reference to the flowcharts of FIGS. 16 and 18, and the schematic diagrams of FIGS. 17, 19 and 20.

Now, it is assumed that steps ST10 to ST30 are executed in the same manner as described above, and the support information table T1, the weight table T2, and the display candidate table T3 are stored in the memory 11.

After step ST30, in step ST40, the processing circuitry 15 creates and updates the optimization target table T5 based on the support information table T1 and the display candidate table T3 in the memory 11, and stores the optimization target table T5 into the memory 11. Such step ST40 is executed, for example, by steps S41 to S47a shown in FIG. 16.

In other words, the processing circuitry 15 acquires the support information table T1 and the display candidate table T3 from the memory 11 (step S41). The processing circuitry 15 rearranges the data in each row in the support information table T1 so that the disease risk is in descending order (step S42). The processing circuitry 15 compares a plurality of medical information pieces for the same disease risk or adjacent disease risks (step S43a), and determines whether a plurality of medical information pieces are integrated, depending on whether they include the same data type and dates within the same period (step S44a). When the plurality of medical information pieces are integrated, the processing circuitry 15 calculates the cost C based on the disease risks and the weights for the plurality of medical information pieces to be integrated, for example, as shown in FIG. 17. Further, the processing circuitry 15 assigns a target ID to the calculated cost C and the optimization target data (the support information ID and the index) for specifying the medical information, adds them to the optimization target table T5 in descending order of the cost C (step S45a), and shifts to step S46. In other words, not only the medical information related to the adjacent disease risk, but also the medical information having common data type and examination period is written in the optimization target table T5 as an optimization target. If the result of the determination in step S44a is negative, the processing proceeds to step S46.

In step S46, the processing circuitry 15 determines whether the processing of step S43a and subsequent steps thereto has been completed for the data in all the rows in the display target table T3, and if the determination is negative, the processing on the data in the unprocessed rows is executed, so that the processing returns to step S43a.

When the processing is completed as a result of the determination in step S46, the processing circuitry 15 compares the display candidate table T3 with the optimization target table T5, and extracts, as medical information pieces to be non-integrated, medical information pieces which are present in the display candidate table T3, but are not present in the optimization target table T5. Further, the processing circuitry 15 calculates the cost C for each medical information piece to be non-integrated based on the disease risk and the weight. Further, the processing circuitry 15 assigns a target ID to the calculated cost C and optimization target data (a support information ID and an index) for specifying the medical information piece, and adds them to the optimization target table T5 in descending order of the cost C (step S47a).

Thereafter, the processing circuitry 15 stores the optimization target table T5 into the memory 11 (step S48a), and ends step S40.

After step ST40, in step ST50, the processing circuitry 15 adjusts the size of the display region of the medical information based on the cost C (the index based on the disease risk and the weight) in the optimization target table T5. Here, the processing circuitry 15 may adjust the size of the display region so that the order of the size corresponds to the order of the size proportional to the disease risk and the weight. For example, the processing circuitry 15 may adjust the size of the display region of the medical information so that the order of the size corresponds to the descending order of the cost C in the optimization target table T5. Such step ST50 is executed, for example, by steps S51a to S55a shown in FIG. 18.

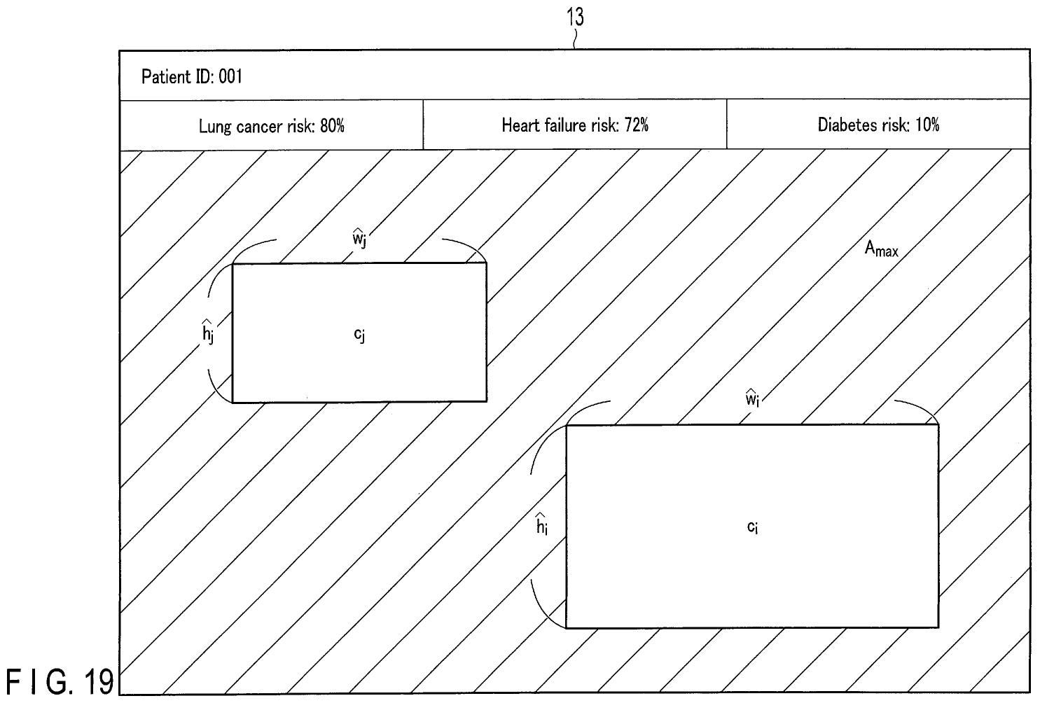

In other words, the processing circuitry 15 acquires the support information table T1, the weight table T2, the display candidate table T3, and the optimization target table T5 from the memory 11 (step S51a). The processing circuitry 15 adjusts the size of the display region of the medical information based on the optimization target table T5 (step S52a). For example, as shown in FIG. 19 and the following formulas (1) to (3), the processing circuitry 15 may adjust the size of the display region, for example, by using a method such as a genetic algorithm (GA).

$$\hat{h} , \hat{w} = {argmax}_{h , w} {\sum }_{i = 1} ( {h}_{i} \times {w}_{i} ) \qquad ( 1 )$$

$$subject to$$

$${C}_{i} \geq {C}_{j} and ( \hat{{h}_{i}} \times {\hat{w}}_{i} ) \geq ( \hat{{h}_{j}} \times {\hat{w}}_{j} ) \qquad ( 2 )$$

$${A}_{max} \geq {\sum }_{i} ( \hat{{h}_{i}} \times {\hat{w}}_{i} ) \qquad ( 3 )$$

In the above formulas, “\hat{ }” attached to each of the height h and the width w of the display region is a hat symbol. Hereinafter, the height h with the hat symbol is expressed as the height \hat{ }h. The height \hat{ }h means an estimated value of the height. Further, the width w with the hat symbol is represented as the width \hat{ }w. The width \hat{ }w means an estimated value of the width. Further, i and j represent identification numbers that can uniquely identify the display regions, and the target ID in the optimization target table T5 is used. In other words, the height hi, the width wi, and the cost Ci are the height h, the width w, and the cost C in the same display region indicated by the identification number i. Further, the height hj, the width wj, and the cost Cj are the height h, the width w, and the cost C in the same display region indicated by the identification number j different from the identification number i. As shown in FIG. 19, Amax represents the area of the entire display region of medical information in the display screen of the display 13. In FIG. 19, two display regions have different heights \hat{ }hi and \hat{ }hj and different widths \hat{ }wi and \hat{ }wj, but the present invention is not limited to this style. For example, the two display regions may have the same height \hat{ }hi and \hat{ }hj, and may have different widths \hat{ }wi and \hat{ }wj. When the heights \hat{ }hi and \hat{ }hj are the same, a plurality of display regions can be easily arranged along the row direction. Alternatively, for example, the two display regions may have different heights \hat{ }hi and \hat{ }hj and have the same widths \hat{ }wi and \hat{ }wj. When the widths \hat{ }wi and \hat{ }wj are the same, a plurality of display regions can be easily arranged along the column direction.

In any case, the processing circuitry 15 adjusts the size of the display region having the height and the width. For example, from the formula (1), the processing circuitry 15 estimates, as \hat{ }h, \hat{ }w, a set of height hi and width wi that maximizes the total area of the display regions represented by the identification number i (i=1, 2, . . . ). However, the processing circuitry estimates \hat{ }h, \hat{ }w according to the constraint conditions shown in the formulas (2) to (3). Here, the formula (2) shows a constraint condition that the area of each display region is in descending order of the cost C. Further, the formula (3) shows a constraint condition that the total area of the display regions is equal to or less than the area Amax of the entire display region.

Thereafter, the processing circuitry 15 determines the arrangement of the display regions of the medical information pieces based on the adjustment result (step S53a). For example, the processing circuitry 15 uses the height \hat{ }h and width \hat{ }w of the display region of each medical information piece to determine the arrangement of each display region so that the display regions shifts from an upper left position to an upper right position, skips from the upper right position to a lower left position, and shifts from the lower left position to a lower right position in descending order of the cost C. In this case, the display region having the maximum cost C is arranged at the upper left position, and the display region having the minimum cost C is arranged at the lower right position. However, the arrangement is not limited to this example.

After step S53a, the processing circuitry 15 compares the display candidate table T3 with the medical information table T2 (step S54a). As a result of the comparison, the processing circuitry 15 determines the arrangement of the medical treatment direction that is not present in the display candidate table T3 (step S55a). After step S55a, the processing circuitry 15 ends the processing of step S50.

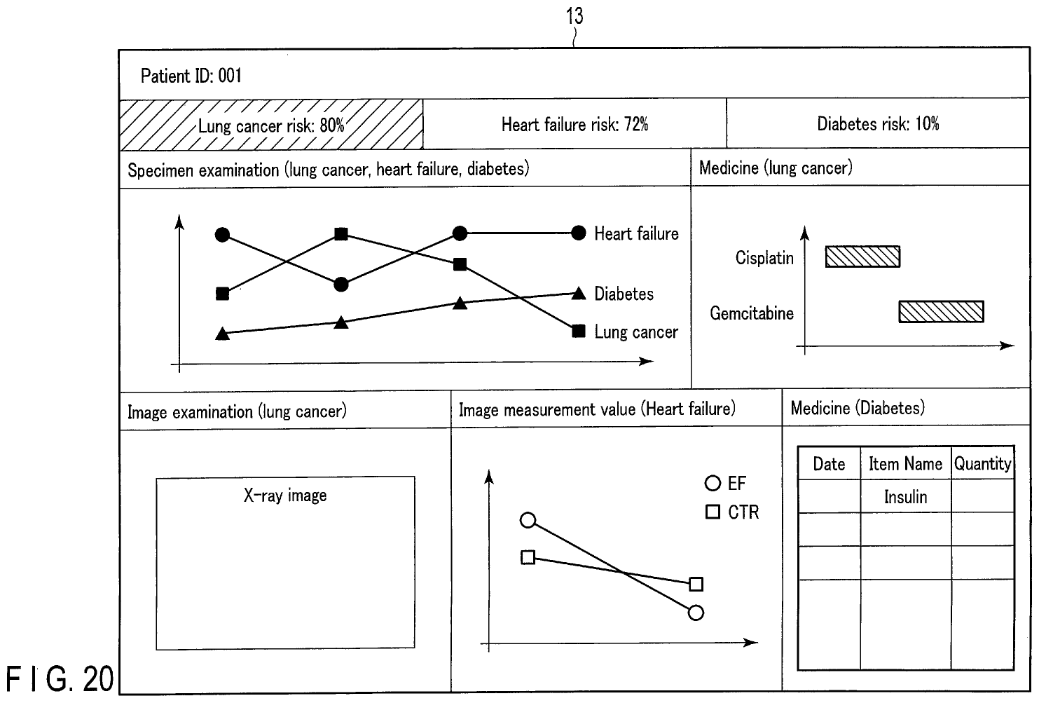

Hereinafter, in step ST60, the processing circuitry 15 controls the display 13 so as to display a plurality of support information pieces and medical information pieces as display candidates based on the determined arrangement. As a result, the display 13 displays a plurality of support information pieces and a plurality of medical information pieces based on the determined arrangement, for example, as shown in FIG. 20. At this time, based on the data type and the period/time of a plurality of medical information pieces associated with a patient ID, the processing circuitry 15 may read out a plurality of actual data of the plurality of medical information pieces from the memory 11, and cause the display 13 to display a screen on which a plurality of support information and the plurality of actual data are arranged. Alternatively, when the arrangement is determined in step ST53a, based on the data type and the period/time of a plurality of medical information pieces associated with a patient ID, the processing circuitry 15 may read out a plurality of actual data of the plurality of medical information pieces from the memory 11, and determine the arrangement by using a plurality of support information and the plurality of actual data. In any case, in the example of FIG. 20, unlike the above-mentioned FIGS. 12 to 14, each of the support information (disease name, disease risk) and the medical information (data type, period/time) is arranged side by side in the column direction. In this arrangement, the support information and the medical information are displayed in association with each other, so that the display region of each medical information piece includes a disease name. However, the present invention is not limited to this style, and both the support information (disease name, disease risk) and the medical information (data type, period/time) may be arranged along the row direction. Alternatively, as in any of FIGS. 12 to 14, the support information and the medical information may be displayed by using directions orthogonal to each other.

As described above, according to the second embodiment, the arrangement is determined by adjusting the size of the display region of medical information pieces to be non-integrated and the size of the display region of medical information pieces to be integrated based on the index values (for example, disease risks) related to diseases and weights. As a result, in addition to the action and effect of the first embodiment, it is possible to adjust the size of the display region of medical information according to the important degree of the medical information.

Further, according to the second embodiment, the size of the display region may be adjusted so that the order of the size corresponds to the order of the size proportional to the index value and the weight associated with the disease. In this case, the more important the medical information is, the larger the area of the display region of the medical information can be made.

According to at least one embodiment described above, each medical information as a display candidate is determined based on a plurality of support information pieces which individually include index values related to a plurality of diseases and the weight of each of a plurality of medical information pieces which is related to each of the index values related to a plurality of diseases. Further, based on respective medical information pieces as display candidates and a plurality of support information, it is determined whether the respective medical information pieces are integrated or not. Further, as a result of the determination, the arrangements of the medical information pieces to be integrated and the medical information pieces to be non-integrated are determined based on a plurality of support information. Therefore, in a limited display range, respective related medical information can be presented to the user for a plurality of support information.

The term of “processor” used in the above description means, for example, a CPU (central processing unit), a GPU (Graphics Processing Unit), or a circuit such as an application specific integrated circuit (ASIC), a programmable logic device (for example, Simple Programmable Logic Device (SPLD)), a complex programmable logic device (CPLD), and a field programmable gate array (FPGA). The processor can implement a function by reading out and executing a program stored in the memory. Instead of storing the program in the memory, the program may be directly incorporated in the circuit of the processor. In this case, the processor implements a function by reading out and executing a program incorporated in the circuit. Note that the processor of the present embodiment is not limited to a case where each processor is configured as a single circuit, and may be applied to a case where a plurality of independent circuits are combined to configure a processor and implement the function thereof. Further, a plurality of components in FIG. 1, 2 or 11 may be integrated into a processor to implement the function thereof.

The medical information processing apparatus as described above may be expressed as shown in the following {0} to {4}. The following expression describes “disease risk” as an example of the index value related to the disease as described above. The index value for the disease is not limited to this example.

{0} A medical information processing apparatus comprises an arrangement determination unit that determines an arrangement for displaying a plurality of support information and medical information forming a basis for the plurality support information on a screen. The arrangement determination unit may determine the arrangement so that a plurality of medical information forming a basis for a plurality of support information (disease risk) can be fit in the screen. For example, the arrangement may be an arrangement in which common portions of the plurality of medical information are integrated so that the plurality of medical information can be fit in a screen. Further, the arrangement may be an arrangement in which medical information which can be fit in a screen out of a plurality of medical information is displayed on an initial screen, and medical information which cannot be not fit in a screen is displayed on an updated screen by updating the initial screen according to a user's operation. Further, the arrangement may be an arrangement in which the size of a display region in which each of a plurality of medical information occupies is adjusted so that a plurality of medical information can be fit in a screen.

{1} When there are a plurality of support information pieces (disease risks) and respective related medical information pieces, a system determines based on each support information piece whether respective medical information pieces can be integrated, and automatically determines the arrangement of the medical information pieces, the system comprising an integration decision/arrangement determination unit. Note that the system may further comprise a display candidate determination unit in addition to the integration decision/arrangement determination unit. Further, the system may comprise other units such as a medical information acquisition unit, a support information/contribution degree calculation unit, and a display unit as appropriate in addition to the display candidate determination unit and the integration decision/arrangement determination unit.

Here, the medical information acquisition unit acquires medical information on a target patient.

The support information/contribution degree calculation unit calculates support information (disease risk) based on a support information calculation model, and calculates the degree of contribution (weight) of medical information (examination information and intervention information and time ranges thereof) contributing to the calculation of the support information.

The display candidate determination unit determines medical information as a display candidate based on support information calculated by a support/basis information calculation unit and the degree of contribution to each medical information.

The integration decision/arrangement determination unit determines whether to integrate medical information pieces as display candidates (cooperative operation) based on the data types, the periods/ranges of the medical information pieces as display candidates, and support information, and determines the arrangements of the medical information pieces as integration targets and the other medical information pieces based on the support information (disease risk) and the degree of contribution.

The display unit displays the information determined by the integration decision/arrangement determination unit on a screen.

{2} In the system described in {1} described above, with respect to medical information pieces for adjacent support information pieces when the support information pieces (disease risks) are set in descending order, in the case of the same data type, near period/time, the integration decision/arrangement determination unit integrates the data thereof.

Here, the integration decision/arrangement determination unit may determine whether integration is possible or not, for example, in the medical information for each support information.

Further, for example, the integration decision/arrangement determination unit may extract common portions in the data type and the period/time to decide whether integration can be performed. Specifically, for example, it may be decided that integration is performed if there are common data type and period/time with respect to medical information for adjacent support information pieces when disease risks are arranged in descending order. If there is a common data type, but there is no adjacent support information, it may be decided that integration is not performed. Further, the number of data types to be integrated may be three or more. However, it is assumed that when the disease risks are arranged in descending order, the corresponding support information pieces are adjacent to each other. Further, the user may designate the granularity of an item as the data type. For example, there are granularities such as specimen examination and image inspection, and granularities of examination item names such as blood pressure and ultrasound. With respect to the period/time, if there are overlapping periods or temporally adjacent periods, they may be determined as common ones. Further, if the difference in period/time is equal to or less than a preset threshold value (when the difference is close to the threshold value to some extent), the integration may be performed. If there is a common portion, medical information pieces (integrated medical information pieces) may be displayed across support information so that an examination item name/period/time to which the support information paid attention may be understood.

On the other hand, when there is no common portion, the integration decision/arrangement determination unit may determine the arrangement of medical information based on support information and an integration decision result. For example, the region of each support information may be determined (arranged in the row direction or the column direction) based on the disease risk. Further, medical information pieces which are extracted in order of contribution degree may be arranged in the determined region. Here, even if medical information pieces have contribution degrees smaller than the threshold value, the medical information pieces may be arranged so as to be displayed in the order of the contribution degree by scrolling.

{3} In the system described in {1} or {2} described above, the integration decision/arrangement determination unit determines the arrangement of various medical information pieces based on the support information (disease risk) and the contribution degree by using a predetermined rule or an optimization method. Giving some supplemental description, the integration decision/arrangement determination unit may optimize a layout based on the data type/contribution degree to each support information, and determine the arrangement of data to be displayed.

Specifically, for example, an optimization target data table is created based on the data type/contribution degree for each support information. At that time, the weight (cost) of each data when optimization is performed may be calculated. Further, data of the same type in the same support information may be added to the optimization target data table in a lump. At that time, with respect to the cost, the average contribution degree may be multiplied by the support information (disease risk). Further, if there is a data type common to a plurality of support information, it may be added to the optimization target data table. The cost may be calculated by determining a weighted average when each contribution degree is multiplied by a disease risk, and multiplying the value of the weighted average by an arbitrary coefficient (0-1). Further, the size of common data may be adjusted by adjusting an arbitrary coefficient.

Further, for example, the height h and the width w of each data type may be optimized so that the height h and the width w of each data type are fit in the screen (area Amax). Note that a restriction may be provided so that the area of the data type having a high cost increases. A method such as a genetic algorithm (GA) may be used for the optimization. Further, the arrangement may be determined based on the height h and the width w of each optimized data.

{4} In the system described in any one of {1} to {3} described above, the display candidate determination unit extracts items that are equal to or more than a preset threshold value in the contribution degree, and sets the extracted data as medical information as a display candidate.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured to:
determine respective medical information pieces as display candidates based on a plurality of support information pieces which individually include index values related to a plurality of diseases, and respective weights of a plurality of medical information pieces related to the respective index values related to the plurality of diseases;
decide whether or not to integrate the respective medical information pieces based on the respective medical information pieces as the display candidates, and the plurality of support information pieces, and
determine an arrangement of medical information pieces to be integrated and an arrangement of medical information pieces to be non-integrated based on the plurality of support information pieces as a result of the decision.

2. The medical information processing apparatus according to claim 1, wherein each of the plurality of medical information pieces includes a data type and a date, and the processing circuitry determines to integrate respective medical information pieces each including an identical data type and dates within an identical period among a plurality of medical information pieces used for calculating adjacent index values when the index values related to the plurality of diseases are arranged side by side in order.

3. The medical information processing apparatus according to claim 1, wherein the processing circuitry determines the arrangement so that the medical information pieces to be non-integrated are arranged side by side along a row or column direction in association with each of the support information pieces, and determines the arrangement so that the medical information pieces to be integrated are arranged side by side in a column or row direction perpendicular to the former direction in associated with a plurality of support information pieces.

4. The medical information processing apparatus according to claim 1, wherein the processing circuitry determines the arrangement by adjusting a size of a display region of the medical information pieces to be non-integrated and a size of a display region of the medical information pieces to be integrated based on the index values and the weights.

5. The medical information processing apparatus according to claim 4, wherein the processing circuitry adjusts the size of the display region so that an order of the size is proportional to the index value and the weight.

6. The medical information processing apparatus according to claim 1, wherein the processing circuitry is further configured to:
acquire a plurality of medical information pieces for a target patient;
based on a calculation model for calculating an index value by summing points obtained by individually scoring a plurality of medical information pieces, calculate respective index values from the acquired plurality of medical information pieces to acquire a plurality of support information pieces which individually include the respective index values, and calculate a ratio of points of each of the acquired plurality of medical information pieces to total points as a weight of each of the medical information pieces; and
determine respective medical information pieces as the display candidates by using the acquired plurality of support information pieces and the respective calculated weights.

7. The medical information processing apparatus according to claim 1, further comprising a memory that stores the index values and respective weights of a plurality of medical information pieces in advance in association with the plurality of medical information pieces, wherein the processing circuitry is configured to:
acquire a plurality of medical information pieces in a target patient;
refer to the memory based on the acquired plurality of medical information pieces to acquire a plurality of support information pieces which individually include the index values related to the plurality of diseases, and respective weights of a plurality of medical information pieces related to the respective index values associated with the plurality of diseases; and
determine the respective medical information pieces as the display candidates by using the acquired plurality of support information pieces and the respective weights.

8. The medical information processing apparatus according to claim 1, wherein the processing circuitry is configured to control a display to display the plurality of support information pieces and the medical information pieces as the display candidates based on the determined arrangements.
